(19) United States
(12) Patent Application Publication
(10) Pub. No.: US 2023/0174671 A1
(43) Pub. Date: Jun. 8, 2023

(54) COMPOSITIONS AND METHODS FOR TREATING VIRAL INFECTIONS

(71) Applicant: BIOVISTA, INC., Charlottesville, VA (US)

(72) Inventors: Eftychia LEKKA, Athens (GR); Aris PERSIDIS, Arvada, CO (US); Christos ANDRONIS, Athens (GR); Vassilis VIRVILIS, Piraeus (GR)

(21) Appl. No.: 17/917,726

(22) PCT Filed: Apr. 9, 2021
(86) PCT No.: PCT/US2021/026646

§ 371 Date: Oct. 7, 2022

Related U.S. Application Data
(60) Provisional application No. 63/007,710, filed on Apr. 9, 2020.
(60) Provisional application No. 63/060,811, filed on Aug. 4, 2020.

(51) Int. Cl. C07K 16/36; A61K 45/06; A61K 31/40; A61K 31/366; A61K 31/505; A61K 31/397; A61P 31/12

(52) U.S. Cl. CPC C07K 16/36; A61K 45/06; A61K 31/40; A61K 31/366; A61K 31/505; A61K 31/397; A61P 31/12; C07K 2317/24; C07K 2317/31; C07K 2317/569

(57) ABSTRACT

Described herein are compounds, compositions, kits, methods, and the uses thereof for treating diseases caused at least in part by virus infection.

### CROSS-REFERENCE TO RELATED APPLICATIONS

This application claims the benefit under 35 U.S.C. § 119(e) of U.S. Provisional Application Ser. Nos. 63/007,710, filed Apr. 9, 2020, and 63/060,811, filed Aug. 4, 2020, the disclosures of which are incorporated herein by reference in its entirety.

### TECHNICAL FIELD

The invention described herein pertains to compounds, compositions, kits, methods, and the uses thereof in treating diseases caused at least in part by virus infection.

### BACKGROUND AND SUMMARY OF THE INVENTION

The 2020-2021 COVID-19 epidemic is a major global concern, affecting over 100 million of people, and resulting in 3 million deaths. The disease has been associated with the SARS-CoV-2 virus infection, affecting countries worldwide and carrying a considerable mortality rate. There is currently no treatment option for COVID-19 or other coronavirus-related infections.

SARS-CoV-2 is a member of the coronavirus family, which shares about 80% identity (Zhou et al, 2020) with that of the Severe Acute Respiratory Syndrome (SARS) coronavirus, SARS-CoV, which was responsible for the 2002-2003 global epidemic. The SARS-CoV-2 is a positive-strand RNA virus that also causes severe respiratory syndrome in humans (Yang et al, 2020). Respiratory failure from acute respiratory distress syndrome (ARDS) is reportedly the primary cause of mortality in COVID-19 patients (Ruan et al, 2020).

Cell entry of the coronaviruses relies on the binding of the viral spike (S) proteins to cellular receptors and on S protein priming by host cell proteases. The biology of SARS-CoV-2 is much less clear than that of SARS-CoV; however, reports suggest that host cell entry of SARS CoV-2 depends on the SARS-CoV receptor ACE2, and the cellular serine protease TMPRSS2, which is employed by SARS-CoV-2 for S protein priming (Hoffman et al, 2020) (Yang et al, 2020) (Wang et al, 2020).

TMPRRSS2 has been shown to play an important role for efficient SARS-CoV-2 entry into the host. TMPRSS2 is a type II transmembrane protease that, similarly to SARS-CoV-2, was previously found to activate the spike (S) protein of the SARS-CoV on the cell surface following receptor binding during viral entry into cells (Kawase et al, 2012). Studies of in vitro SARS-CoV models have pointed toward a possible beneficial role of TMPRSS2 blockade for the management of coronavirus infections. In a relevant study model, camostat mesylate, a TMPRSS2 inhibitor, was able to partially block infection by SARS-CoV and human coronavirus NL63 (HCoV-NL63) in HeLa cells expressing TMPRSS2 (Kawase et al, 2012). TMPRSS2 inhibition has previously been found to confer protection against other types of lethal virally-induced pneumonias (Hayashi et al, 1991). Though without being bound by theory, due to the similar role that TMPRSS2 plays in the host entry also in the case of SARS-CoV-2, it is believed herein that blockade of TMPRSS2 will be therapeutically effective in treating diseases caused by SARS-CoV-2, such as COVID-19.

Alongside TMPRRSS2, the Angiotensin Converting Enzyme 2 Receptor (ACE2) has been shown to play an important role for efficient SARS-CoV-2 entry into the host. ACE2 is a homologue of the ACE receptor dipeptidyl carboxydipeptidase. ACE2 is expressed in the lungs, stomach, spleen, intestine, bone-marrow, kidney, liver, and the brain (Gembardt, et al, 2005) (Soler et al, 2008). In the lungs, ACE2 is expressed on the vascular endothelium, type I and type II alveolar epithelial cells, the smooth muscle cells of the pulmonary vasculature, and in bronchial epithelia (Hamming et al, 2004). Yet, despite the homology between the ACE receptor and ACE2, these two molecules serve opposing physiological functions with respect to the regulation of the renin-angiotensin system (RAS). The RAS plays a critical role in cardiovascular disorders, in controlling blood pressure, and body fluid and electrolyte homeostasis. Although the ACE/Ang-II/AT1R system is a well-established axis of the RAS leading to vasoconstrictive, fibrotic and proliferative effects, ACE2 linking to angiotensin (1-7) [Ang(1-7)] and its G-protein coupled protein receptor Mas, exhibits a vasoprotective, anti-inflammatory and anti-proliferative mechanism, resulting in counter regulation of RAS (Santos et al. 2003) (Chamsi-Pasha et al, 2014).

Studies in animal models of hypertension, as well as hypertensive patients have shown that the administration of certain angiotensin 1 receptor (AT1R) antagonists can result in the up-regulation of ACE2 levels both in animal models (Ishiyama et al., 2004) Klimas et al, 2015) and in human patients (Furuhashi et al., 2015). AT1R stimulation by angiotensin has also been shown to result in increased pulmonary vascular permeability and increased lung pathology (Imai et al, 2005) (Kuba et al, 2005). These observations have led to an hypothesis that chronic administration of AT1R antagonists might lead in the up-regulation of ACE2 levels; thus, it has been suggested by certain groups that hypertensive patients that were on angiotensin receptor blocking drugs (ARBs) would be at higher risk to develop SARS-CoV-2 infections (Esler and Esler, 2020) leading to severe and fatal COVID-19 (Fang et al, 2020).

The foregoing suggests that angiotensin receptor blockers would be detrimental to COVID-19 patients. However, it has been discovered that upon binding of the coronavirus spike protein to ACE2 receptor, ACE2 is unexpectedly down-regulated and results in excessive production of angiotensin by ACE enzyme, and eventually leads to a diminished amount of ACE2 converting into the vasodilator heptapeptide angiotensin 1-7. That mechanism may thus contribute to lung injury. Therefore, though without being bound by theory, it is believed herein that high ACE2 expression induced by ARBs could in fact protect patients against acute lung injury by ultimately increasing the production of the vasodilator angiotensin 1-7.

It has also been reported that the risk of hospitalization, morbidity, and mortality from COVID-19 is highest for older patients with preexisting conditions such as hypertension, diabetes, cardiovascular disease and obesity. It is understood herein that a common feature of these comorbidities is their association with vascular inflammation and endothelial dysfunction. Without being bound by theory, it is observed that endothelial cells express ACE2, and therefore, it is believed herein that such expression may mediate endothelial activation upon viral infection.

Altered coagulation is also a common feature of acute systemic diseases, specifically to those affecting the respiratory system. Reportedly, there is a consistent picture of altered coagulation in patients with severe COVID-19, as well as a higher incidence of thrombotic events, particularly pulmonary embolism in COVID-19 patients (reviewed in Katneni et al, 2020). Several groups have already reported that patients infected with SARS-CoV-2 admitted to the ICU show a high rate of thromboembolic complications ranging between 25% and 35% (Beun et al, 2020) (Klok et al, 2020) (Cui et al, 2020). These patients have a hypercoagulable state at admission to the ICU and show mainly renal impairment as second organ involvement (Huisman et al, 2020).

Without being bound by theory, it is believed herein that these severe events may be caused by endothelial dysfunction in COVID-19, which resembles Thrombotic Microangiopathy (TMA). TMA is observed as a consequences of sepsis and may be caused by greatly reduced levels of a disintegrin-like metalloprotease with a thrombospondin type 1 motif, member 13 (ADAMTS13) which is a von Willebrand (vWF) cleaving protease. Again, without being bound by theory, it is believed herein that severe complications in COVID-19 are a result of a disequilibrium state between insufficient ADAMTS13 and excessive exocytosis of ultra large von Willebrand Factor multimers (ULvWF) from Weibel-Palade bodies present in endothelial cells due to COVID-19-induced endotheliopathy (see, for example, Varatharajah et al, 2020). It has been discovered herein that low ADAMTS13 activity and high vWF levels identified in SARS-CoV2 patients support the conclusion that the microangiopathic state observed in these patients is a result of an imbalance in this pathway. Also, there have been reports for thrombotic thrombocytopenic purpura (TTP), a disease with a hallmark vWF/ADAMTS13 imbalance, associated with COVID-19 (Albiol et al, 2020). In addition to the thrombotic events, this imbalance could also be responsible for severe ARDS and multi-organ dysfunction seen in COVID-19 patients, leading mostly to fatal outcomes.

It is understood herein that von Willebrand Factor (vWF) and ADAMTS13 interactions play an important role in the maintenance of hemostasis and prevention of unwanted thrombosis. In inflammatory conditions, vWF-ADAMTS13 imbalance characterized by elevated vWF levels and inhibited and/or reduced activity of ADAMTS13 is reported. Also, an imbalance between ADAMTS13 activity and vWF antigen is associated with organ dysfunction and death in patients with systemic inflammation.

It has also been discovered herein that modulating the vWF-ADAMTS13 imbalance may provide clinically beneficial outcomes in severe COVID-19 cases characterized by excessive release of vWF in endothelial cells, and concomitant microthrombotic complications that are frequently observed in COVID-19 severe cases.

It has also been discovered herein that two classes of drugs are useful in treating diseases caused at least in part by coronavirus infections and other enveloped viruses. In one illustrative embodiment of the invention described herein, the compounds, compositions, methods, and uses described herein include compounds capable of increasing ACE2 production and/or expression. Without being bound by theory, it is believed herein that such increased production can mediate the increases in vulnerability to the damaging effects of AT-II, which is believed herein to be a major cause of lung injury. In addition, without being bound by theory, it is believed herein that such drugs can ameliorate inflammation, including vascular inflammation, and endothelial dysfunction.

Illustrative compounds for augmenting ACE2 include, but are not limited to, cholesterol-lowering agents, HMG-CoA reductase inhibitors, and statins, such as atorvastatin, simvastatin, rosuvastatin, and the like. The observed strong anti-inflammatory effects of statins, such as atorvastatin, may increase ACE2 production and expression, as well as inhibit the Toll-like receptor (TLR)-MYD88-NFκB pathway (see, for example, Chansrichavala et al, 2009). More specifically, administering statins, such as atorvastatin, may lower CRP in a manner independent of low-density lipoprotein cholesterol (LDL-C). Without being bound by theory, it is believed herein that control of inflammation is important for COVID-19 patients, as anti-inflammatory activity might improve outcomes of patients with severe illness, severe respiratory failure, and increasing D-dimer and IL-6 levels; these parameters have been shown to associate with increased mortality.

Illustrative compounds also include, but are not limited to, cholesterol absorption inhibitors, such as ezetimibe, and the like. In addition, combinations of cholesterol-lowering agents and cholesterol absorption inhibitors, such as statins and ezetimibe, are also described herein.

In another illustrative embodiment of the invention described herein, the compounds, compositions, methods, and uses described herein include compounds capable of targeting vWF, such as the human vWF A1 domain and inhibiting vWF-platelets interactions. Illustrative compounds also include, but are not limited to, antibody fragments, such as caplacizumab (CABLIVI™). Caplacizumab is a humanized single-variable domain immunoglobulin that recognizes the human vWF A1 domain and inhibits the vWF-platelet GP1b-αinteraction. Without being bound by theory, it is believed herein that compounds capable of targeting vWF, such caplacizumab, are useful in treating thrombotic events comorbid with viral infections. For example, caplacizumab is useful in treating acquired thrombotic thrombocytopenic purpura (aTTP). In another embodiment, compounds capable of targeting vWF, such as caplacizumab, are used in combination with plasma exchange and immunosuppressive therapy.

In another embodiment, the compounds, compositions, methods, and uses described herein include certain vitamins, such as a vitamin D, and the administration thereof. In another embodiment, the compounds, compositions, methods, and uses described herein include certain antioxidant compounds, such as lycopene, and the administration thereof.

It has also been discovered that compounds such as a vitamin D and/or lycopene are useful in managing the manifestations of diseases caused by virus infection. In another embodiment, the compounds, compositions, methods, and uses described herein include a vitamin D as a co-therapy for use with other compounds described herein. In another embodiment, the compounds, compositions, methods, and uses described herein include lycopene as a co-therapy for use with other compounds described herein.

The compounds, compositions, methods, and uses described herein are useful for treating such viral infections, including infections caused by enveloped viruses such as Coronaviridae.

In another illustrative embodiment, pharmaceutical compositions containing one or more of the compounds are also described herein. In another embodiment, the compositions include a therapeutically effective amount of the one or more compounds for treating a host animal with a viral infection. It is to be understood that the compositions may include other components and/or ingredients, including, but not limited to, other therapeutically active compounds, and/or one or more carriers, diluents, excipients, and the like, and combinations thereof.

In another embodiment, methods for treating a host animal with a viral infection are also described herein, where the methods include administering one or more of the compounds and/or compositions described herein to the host animal. In another embodiment, the methods include administering a therapeutically effective amount of the one or more compounds and/or compositions described herein for treating host animals with a viral infection. In another embodiment, uses of the compounds and compositions in the manufacture of a medicament for treating host animals with a viral infection are also described herein. In another embodiment, the medicaments include a therapeutically effective amount of the one or more compounds and/or compositions for treating a host animal with a viral infection.

It is to be understood herein that the compounds and compositions, and methods described herein may be used alone or in combination with other compounds useful for treating viral infections, including those compounds that may be therapeutically effective by the same or different modes of action. In addition, it is to be understood herein that the compounds described herein may be used in combination with other compounds that are administered to treat other symptoms of a viral infection, such as compounds administered for treating pain, inflammation, shortness of breath, congestion, fever, and the like.

### DETAILED DESCRIPTION

Several illustrative embodiments of the invention are described by the following clauses:

A composition for treating a viral infection, the composition comprising one or more cholesterol-lowering agents, cholesterol absorption inhibitors, or a combination thereof.

A composition for treating a viral infection, the composition consisting essentially of one or more cholesterol-lowering agents, cholesterol absorption inhibitors, or a combination thereof.

The composition of any one of the preceding clauses wherein the cholesterol-lowering agent is atorvastatin, simvastatin, or rosuvastatin, or a salt of the foregoing.

The composition of the preceding clauses wherein the cholesterol-lowering agent is atorvastatin.

The composition of the preceding clause wherein the cholesterol absorption inhibitors is ezetimibe.

The composition of any one of the preceding clauses wherein the combination of cholesterol-lowering agents and cholesterol absorption inhibitor is atorvastatin/ezetimibe.

A composition for treating a viral infection, the composition comprising one or more compounds capable of targeting vWF and inhibiting vWF-platelet interaction.

A composition for treating a viral infection, the composition consisting essentially of one or more compounds capable of targeting vWF and inhibiting vWF-platelet interaction.

The composition of any one of the preceding clauses wherein the compound capable of targeting vWF and inhibiting vWF-platelet interaction is caplacizumab.

The composition of any one of the preceding clauses wherein the composition further comprises one or more angiotensin receptor blockers, including angiotensin II receptor blockers.

The composition of any one of the preceding clauses wherein the composition further comprises one or more protease inhibitors.

The composition of any one of the preceding clauses wherein the composition further comprises one or more protease inhibitors selected from the group consisting of aprotinin, laskowski inhibitors, lympho-epithelial kazal-type-related inhibitor (lekti), leupeptin, camostat, β-conglycinin, aptivus (tipranavir), reyataz (atazanavir), crixivan, idv (indinavir), prezista (darunavir), lexiva (fosamprenavir), invirase (saquinavir), kaletra (lopinavir/ritonavir), viracept (nelfinavir), norvir (ritonavir), and salts thereof.

The composition of any one of the preceding clauses wherein the composition further comprises one or more protease inhibitors selected from the group consisting of irbesartan, azilsartan (Edarbi), candesartan (Atacand), eprosartan (Teveten), irbesartan (Avapro), telmisartan (Micardis), valsartan (Diovan, Prexxartan), losartan (Cozaar), olmesartan (Benicar), entresto (sacubitril/valsartan), byvalson (nebivolol/valsartan), and salts thereof.

The composition of any one of the preceding clauses wherein the protease inhibitor is aprotinin, or a salt thereof.

The composition of any one of the preceding clauses wherein the protease inhibitor is irbesartan, or a salt thereof.

The composition of any one of the preceding clauses wherein the composition further comprises one or more vitamins, one or more antioxidants, or a combination of the foregoing.

The composition of any one of the preceding clauses wherein the composition further one or more antioxidants.

The composition of any one of the preceding clauses wherein the composition further comprises lycopene.

A kit for treating a viral infection, the kit comprising a composition of any one of the preceding clauses; and instructions for administering the pharmaceutically active ingredients therein.

A kit for treating a viral infection, the kit consisting essentially of a composition of any one of the preceding clauses; and instructions for administering the pharmaceutically active ingredients therein.

Use of one or more cholesterol-lowering agents, cholesterol absorption inhibitors, or a combination thereof in the manufacture of a medicament for treating a viral infection.

Use of one or more compounds capable of targeting vWF and/or inhibiting vWF-platelet interaction in the manufacture of a medicament for treating a viral infection.

A method for treating a viral infection in a host animal, the method comprising administering to the host animal a therapeutically effective amount of one or more cholesterol-lowering agents, cholesterol absorption inhibitors, or a combination thereof.

A method for treating a viral infection in a host animal, the method comprising administering to the host animal a composition consisting essentially of a therapeutically effective amount of one or more cholesterol-lowering agents, cholesterol absorption inhibitors, or a combination thereof.

A method for treating a viral infection in a host animal, the method comprising administering to the host animal a therapeutically effective amount of one or more compounds capable of targeting vWF and/or inhibiting vWF-platelet interaction.

A method for treating a viral infection in a host animal, the method comprising administering to the host animal a composition consisting essentially of a therapeutically effective amount of one or more compounds capable of targeting vWF and/or inhibiting vWF-platelet interaction.

The method of the preceding clauses further comprising administering to the host animal one or more angiotensin receptor blockers, including angiotensin II receptor blockers, one or more protease inhibitors, or a combination thereof.

The method of any one of the preceding clauses wherein the composition further comprises one or more protease inhibitors selected from the group consisting of aprotinin, laskowski inhibitors, lympho-epithelial kazal-type-related inhibitor (lekti), leupeptin, camostat, β-conglycinin, aptivus (tipranavir), reyataz (atazanavir), crixivan, idv (indinavir), prezista (darunavir), lexiva (fosamprenavir), invirase (saquinavir), kaletra (lopinavir/ritonavir), viracept (nelfinavir), norvir (ritonavir), and salts thereof.

The method of any one of the preceding clauses wherein the composition further comprises one or more protease inhibitors selected from the group consisting of irbesartan, azilsartan (Edarbi), candesartan (Atacand), eprosartan (Teveten), irbesartan (Avapro), telmisartan (Micardis), valsartan (Diovan, Prexxartan), losartan (Cozaar), olmesartan (Benicar), entresto (sacubitril/valsartan), byvalson (nebivolol/valsartan), and salts thereof.

The method of any one of the preceding clauses wherein the protease inhibitor is aprotinin, or a salt thereof.

The method of any one of the preceding clauses wherein the protease inhibitor is irbesartan, or a salt thereof.

The method of the preceding clauses further comprising administering to the host animal one or more vitamins, one or more antioxidants, or a combination thereof.

The composition, kit, use, or method of any one of the preceding clauses wherein the vitamin is a vitamin D or an analog or derivative thereof.

The composition, kit, use, or method of any one of the preceding clauses wherein the antioxidant is lycopene or an analog or derivative thereof.

The composition, kit, use, or method of the any one of the preceding clauses wherein the virus is an enveloped virus.

The composition, kit, use, or method of any one of the preceding clauses wherein the virus is a RNA virus.

The composition, kit, use, or method of any one of the preceding clauses wherein the virus is a positive-sense virus.

The composition, kit, use, or method of any one of the preceding clauses wherein the virus is a single stranded virus.

The composition, kit, use, or method of any one of the preceding clauses wherein the virus is a Coronaviridae.

The composition, kit, use, or method of any one of the preceding clauses wherein the virus is a Orthocoronavirinae.

The composition, kit, use, or method of any one of the preceding clauses wherein the virus uses, at least in part, host cell ACE2 receptors for entry.

The composition, kit, use, or method of any one of the preceding clauses wherein the virus uses, at least in part, host cell serine protease, such as TMPRSS2, for entry and/or infection.

In each of the foregoing and each of the following embodiments, unless otherwise indicated, it is also to be understood that the formulae include and represent any and all crystalline forms, partially crystalline forms, and non-crystalline and/or amorphous forms of the compounds.

In each of the foregoing and each of the following embodiments, unless otherwise indicated, it is also to be understood that the transitional phrase “consisting essentially of” means that the scope of the corresponding composition, kit, method, or use is to be interpreted to encompass the specified recited compounds and materials and/or steps, and also optionally include additional compounds and materials and/or steps those that do not materially affect the basic and novel characteristics of the claimed invention.

Illustrative derivatives include, but are not limited to, both those compounds that may be synthetically prepared from the compounds described herein, as well as those compounds that may be prepared in a similar way as those described herein, but differing in the selection of starting materials. It is to be understood that such derivatives may include prodrugs of the compounds described herein, compounds described herein that include one or more protection or protecting groups, including compounds that are used in the preparation of other compounds described herein.

The compounds described herein may contain one or more chiral centers, or may otherwise be capable of existing as multiple stereoisomers. It is to be understood that in one embodiment, the invention described herein is not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

It is to be understood that in every instance disclosed herein, the recitation of a range of integers for any variable describes the recited range, every individual member in the range, and every possible subrange for that variable. For example, the recitation that n is an integer from 0 to 8, describes that range, the individual and selectable values of 0, 1, 2, 3, 4, 5, 6, 7, and 8, such as n is 0, or n is 1, or n is 2, etc. In addition, the recitation that n is an integer from 0 to 8 also describes each and every subrange, each of which may for the basis of a further embodiment, such as n is an integer from 1 to 8, from 1 to 7, from 1 to 6, from 2 to 8, from 2 to 7, from 1 to 3, from 2 to 4, etc.

As used herein, the term “composition” generally refers to any product comprising the specified ingredients in the specified amounts, as well as any product which results, directly or indirectly, from combinations of the specified ingredients in the specified amounts. It is to be understood that the compositions described herein may be prepared from isolated compounds described herein or from salts, solutions, hydrates, solvates, and other forms of the compounds described herein. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. Accordingly, such pharmaceutical compositions that recite compounds described herein are to be understood to include each of, or any combination of, the various morphological forms and/or solvate or hydrate forms of the compounds described herein. In addition, it is to be understood that the compositions may be prepared from various co-crystals of the compounds described herein.

Illustratively, compositions may include one or more carriers, diluents, and/or excipients. The compounds described herein, or compositions containing them, may be formulated in a therapeutically effective amount in any conventional dosage forms appropriate for the methods described herein. The compounds described herein, or compositions containing them, including such formulations, may be administered by a wide variety of conventional routes for the methods described herein, and in a wide variety of dosage formats, utilizing known procedures (see generally, Remington: The Science and Practice of Pharmacy, (21^{st} ed., 2005)).

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

It is also appreciated that the therapeutically effective amount, whether referring to monotherapy or combination therapy, is advantageously selected with reference to any toxicity, or other undesirable side effect, that might occur during administration of one or more of the compounds described herein. Further, it is appreciated that the co-therapies described herein may allow for the administration of lower doses of compounds that show such toxicity, or other undesirable side effect, where those lower doses are below thresholds of toxicity or lower in the therapeutic window than would otherwise be administered in the absence of a cotherapy.

In addition to the illustrative dosages and dosing protocols described herein, it is to be understood that an effective amount of any one or a mixture of the compounds described herein can be readily determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “administering” as used herein includes all means of introducing the compounds and compositions described herein to the host animal, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically-acceptable carriers, adjuvants, and/or vehicles.

In making the pharmaceutical compositions of the compounds described herein, a therapeutically effective amount of one or more compounds in any of the various forms described herein may be mixed with one or more excipients, diluted by one or more excipients, or enclosed within such a carrier which can be in the form of a capsule, sachet, paper, or other container. Excipients may serve as a diluent, and can be solid, semi-solid, or liquid materials, which act as a vehicle, carrier or medium for the active ingredient. Thus, the formulation compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders. The compositions may contain anywhere from about 0.1% to about 99.9% active ingredients, depending upon the selected dose and dosage form.

Caplacizumab is a novel monoclonal antibody targeting vWF that is approved for the treatment of TTP and thrombosis. Caplacizumab targets the A1-domain of vWF, inhibiting the interaction between vWF and platelets providing a sustained suppression of the vWF activity (Sargentini-Maier et al, 2019). Without being bound by theory, it is believed herein that by inhibiting the vWF-platelet glycoprotein-Ib interaction, caplacizumab blocks the adhesion of platelets to vWF multimers preventing the formation of the pathological microthrombi, and thus preventing end-organ ischemic damage. As the imbalance between vWF and ADAMTS13 is asserted herein to be a potential cause of the micro-thrombotic events seen in severe COVID-19, caplacizumab is useful for providing management of the thrombotic complications in COVID-19 patients.

Case studies in patients showing milder symptoms of diseases caused by SARS-CoV have shown that these patients mount a robust adaptive immune response as there seems to be a recruitment of immune cell populations (antibody-secreting cells, follicular helper T cells and activated CD4+ and CD8+ T cells) before the resolution of symptoms. Without being bound by theory, it is believed herein that those immune parameters may be exploited in order to confer protection against the virus and help alleviate the inflammatory state seen in COVID-19 patients because the viral infection in COVID-19 patients may also progress in hyper-inflammation and cytokine release syndrome (CRS), also referred to as a cytokine storm. Therefore, without being bound by theory, it is also believed herein that irbesartan is therapeutically effective by at least conferring potent anti-inflammatory and anti-fibrotic properties that will reduce the potential for a cytokine storm common to viral diseases, such as COVID-19.

In addition, SARS-CoV-2 infection has been shown to proceed into acute respiratory distress syndrome (ARDS) with the simultaneous down-regulation of ACE2. (Ferrario et al, 2005) (Gu et al, 2016) (Kuba et al, 2005). Irbesartan, (Aprovel, Karvea, Avapro et al.) is a generic oral medication approved for the treatment of high blood pressure, heart failure, and diabetic kidney disease. Irbesartan is a potent ARB inhibitor that is typically prescribed for the initial management of hypertension. Without being bound by theory, it is believed herein that irbesartan is therapeutically effective by at least up-regulating ACE2. Therefore, the up-regulation of ACE2 that degrades angiotensin II to angiotensin, is believed to improve outcomes in ARDS, SARS, COVID19, and related diseases.

The effective use of the compounds, compositions, and methods described herein for treating or ameliorating one or more effects of a viral infection using one or more compounds described herein may be based upon animal models, such as murine, canine, porcine, and non-human primate animal models of disease.

The following examples further illustrate specific embodiments of the invention; however, the following illustrative examples should not be interpreted in any way to limit the invention.

### EXAMPLES

EXAMPLE. Viral RNA replication in vitro assay. Using a conventional qPCR and/or RT-qPCR assay for RNA expression, for example, commercially available assays from Promega, the compounds and compositions described herein show knock-down of viral RNA expression and signaling.

EXAMPLE. Viral infection in vitro assay. Using a conventional assay for viral infection and host cell entry, for example in monkey or human cells, the compounds and compositions described herein show delayed and/or decreased infection. Briefly, human bronchial epithelial cells, such as Calu-3 cells, are evaluated as described by Beaulieu A. et al. J Virol. 2013 April; 87(8):4237-51. Calu-3 cells are washed with Dulbecco's phosphate-buffered saline (D-PBS) and exposed to test virus strains (diluted in incomplete medium; 0.2% bovine serum albumin [BSA] instead of FBS). After virus adsorption (1 h at 37° C.), cells are washed once with D-PBS, and then incubated in incomplete culture medium containing increasing concentrations of one or more compounds or compositions described herein for 48 h. viral titers may be determined in the supernatants of infected cells by any conventional method, such as by using a viral plaque assay.

EXAMPLE. Viral infection in vitro assay. Using a conventional assay for cell survival, the compounds and compositions described herein show increased cell survival in the presence of replicating virus compared to untreated control cells.

EXAMPLE. Viral infection in vivo assay. Host animals, such as mice (for example, female or male BALB/c mice 8-10 weeks old (Jackson Laboratory, Bar Harbor, Me.), rats, or monkeys are exposed to viral particles. Host animals are separated in treatment and untreated control groups. Compounds and compositions described herein are administered to the treatment groups are various doses. Host animals are assessed for viral load after a predetermined period of time. The compounds and compositions described herein show decreased viral load in a dose dependent manner compared to control animals.

The following publications, and each of the additional publications cited herein are incorporated herein by reference:

Zhou P, Yang X L, Wang X G, Hu B, Zhang L, Zhang W, Si H R, Zhu Y, Li B, Huang C L, Chen H D, Chen J, Luo Y, Guo H, Jiang R D, Liu M Q, Chen Y, Shen X R, Wang X, Zheng X S, Zhao K, Chen Q J, Deng F, Liu L L, Yan B, Zhan F X, Wang Y Y, Xiao G F, Shi Z L. 2020. A pneumonia outbreak associated with a new coronavirus of probable bat origin. Nature. March; 579(7798):270-273.

Yan R, Zhang Y, Li Y, Xia L, Guo Y, Zhou Q. 2020. Structural basis for the recognition of the SARS-CoV-2 by full-length human ACE2. Science. March 4. pii: eabb2762.

Ruan Q, Yang K, Wang W, Jiang L, Song J. 2020. Clinical predictors of mortality due to COVID-19 based on an analysis of data of 150 patients from Wuhan China. Intensive Care Med. March 3. doi: 10.1007/s00134-020-05991-x.

Hoffmann M, Kleine-Weber H, Schroeder S, Krüger N, Herrler T, Erichsen S, Schiergens T S, Herrler G, Wu N H, Nitsche A, Müller M A, Drosten C, Pöhlmann S. 2020. Cell. March 4. pii: 50092-8674(20)30229-4.

Wang Y, Shang J, Graham R, Baric R. S, Li F. 2020. Receptor Recognition by the Novel Coronavirus from Wuhan: an Analysis Based on Decade-Long Structural Studies of SARS Coronavirus Journal of Virology March, 94 (7) e00127-20.

Gembardt F, Sterner-Kock A, Imboden H, Spalteholz M, Reibitz F, Schultheiss H P, Siems W E, Walther T. 2005. Organ-specific distribution of ACE2 mRNA and correlating peptidase activity in rodents. Peptides. July; 26(7):1270-7.

Soler M J, Barrios C, Oliva R, Batlle D. 2008. Pharmacologic modulation of ACE2 expression. Curr Hypertens Rep. October; 10(5):410-4.

Hamming I, Timens W, Bulthuis M L, Lely A T, Navis G, van Goor H. 2004. Tissue distribution of ACE2 protein, the functional receptor for SARS coronavirus. A first step in understanding SARS pathogenesis. J Pathol. June; 203(2):631-7.

Santos R A, Simoes e Silva A C, Maric C, Silva D M, Machado R P, de Buhr I, Heringer-Walther S, Pinheiro S V, Lopes M T, Bader M, Mendes E P, Lemos V S, Campagnole-Santos M J, Schultheiss H P, Speth R, Walther T. 2003. Angiotensin-(1-7) is an endogenous ligand for the G protein-coupled receptor Mas. Proc Natl Acad Sci USA. July 8; 100(14):8258-63.

Chamsi-Pasha M A, Shao Z, Tang W H. 2014. Angiotensin-converting enzyme 2 as a therapeutic target for heart failure. Curr Heart Fail Rep. March; 11(1):58-63.

Ishiyama Y, Gallagher P E, Averill D B, Tallant E A, Brosnihan K B, Ferrario C M. 2004. Upregulation of angiotensin-converting enzyme 2 after myocardial infarction by blockade of angiotensin II receptors. Hypertension. May; 43(5):970-6.

Klimas J, Olvedy M, Ochodnicka-Mackovicova K, Kruzliak P, Cacanyiova S, Kristek F, Krenek P, Ochodnicky P. 2015. Perinatally administered losartan augments renal ACE2 expression but not cardiac or renal Mas receptor in spontaneously hypertensive rats. J Cell Mol Med. August; 19(8):1965-74.

Furuhashi M, Moniwa N, Mita T, Fuseya T, Ishimura S, Ohno K, Shibata S, Tanaka M, Watanabe Y, Akasaka H, Ohnishi H, Yoshida H, Takizawa H, Saitoh S, Ura N, Shimamoto K, Miura T. 2015. Urinary angiotensin-converting enzyme 2 in hypertensive patients may be increased by olmesartan, an angiotensin II receptor blocker. Am J Hypertens. January; 28(1):15-21.

Imai Y, Kuba K, Rao S, Huan Y, Guo F, Guan B, Yang P, Sarao R, Wada T, Leong-Poi H, Crackower M A, Fukamizu A, Hui C C, Hein L, Uhlig S, Slutsky A S, Jiang C, Penninger J M. 2005. Angiotensin-converting enzyme 2 protects from severe acute lung failure. Nature. July 7;436(7047):112-6.

Kuba K, Imai Y, Rao S, Gao H, Guo F, Guan B, Huan Y, Yang P, Zhang Y, Deng W, Bao L, Zhang B, Liu G, Wang Z, Chappell M, Liu Y, Zheng D, Leibbrandt A, Wada T, Slutsky AS, Liu D, Qin C, Jiang C, Penninger J M. 2005. A crucial role of angiotensin converting enzyme 2 (ACE2) in SARS coronavirus-induced lung injury. Nat Med. August; 11(8):875-9.

Esler M, Esler D. 2020. Can angiotensin receptor-blocking drugs perhaps be harmful in the COVID-19 pandemic? J Hypertens. March 11.

Fang L, Karakiulakis G, Roth M. 2020. Are patients with hypertension and diabetes mellitus at increased risk for COVID-19 infection? Lancet Respir Med.

Kawase M, Shirato K, van der Hoek L,Taguchi F, Matsuyama S. 2012. Simultaneous treatment of human bronchial epithelial cells with serine and cysteine protease inhibitors prevents severe acute respiratory syndrome coronavirus entry. J Virol. June; 86(12): 6537-45.

Hayashi T, Hotta H Itoh M Homma M. 1991. Protection of mice by a protease inhibitor, aprotinin, against lethal Sendai virus pneumonia. J Gen Virol April; 72 (Pt 4):979-82.

Thevarajan, I., Nguyen, T. H. O., Koutsakos, M. et al. 2020. Breadth of concomitant immune responses prior to patient recovery: a case report of non-severe COVID-19. Nat Med. https://doi.org/10.1038/s41591-020-0819-2

Mehta P, McAuley D F, Brown M, Sanchez E, Tattersall R S, Manson J J, HLH Across Speciality Collaboration, UK. 2020. COVID-19: consider cytokine storm syndromes and immunosuppression. Lancet March 16. pii: S0140-6736(20)30628-0. doi: 10.1016/S0140-6736(20)30628-0.

Yamaya M1, Shimotai Y2, Hatachi Y3, Lusamba Kalonji N4, Tando Y, Kitajima Y, Matsuo K, Kubo H, Nagatomi R, Hongo S, Homma M, Nishimura H. 2015. The serine protease inhibitor camostat inhibits influenza virus replication and cytokine production in primary cultures of human tracheal epithelial cells. Pulm Pharmacol Ther. August; 33:66-74. doi: 10.1016/j.pupt.2015.07.001.

Iwata-Yoshikawa N, Okamura T, Shimizu Y, Hasegawa H, Takeda M, Nagata N. 2019. TMPRSS2 Contributes to Virus Spread and Immunopathology in the Airways of Murine Models after Coronavirus Infection. J Virol. March 5; 93(6).

Shen L W, Mao H J, Wu Y L, Tanaka Y, Zhang W. 2017. TMPRSS2: A potential target for treatment of influenza virus and coronavirus infections. Biochimie. November; 142:1-10. doi: 10.1016/j.biochi.2017.07.016.

Tanaka J, Tajima S, Asakawa K, Sakagami T, Moriyama H, Takada T, Suzuki E, Narita I. 2013. Preventive effect of irbesartan on bleomycin-induced lung injury in mice. Respir Investig. June; 51(2):76-83.

Ferrario C M, Jessup J, Chappell M C, Averill D B, Brosnihan K B, Tallant E A, Diz D I, Gallagher P E. 2005. Effect of angiotensin-converting enzyme inhibition and angiotensin II receptor blockers on cardiac angiotensin-converting enzyme 2. Circulation; 111:2605-2610.

Gu H, Xie Z, Li T, Zhang S, Lai C, Zhu P, Wang K, Han L, Duan Y, Zhao Z, et al. 2016. Angiotensin-converting enzyme 2 inhibits lung injury induced by respiratory syncytial virus. Sci Rep.; 6:19840.

What is claimed is:

1.-15. (canceled)

16. A method for treating a viral infection in a host animal, the method comprising administering to the host animal a composition comprising a therapeutically effective amount of one or more cholesterol-lowering agents, one or more cholesterol absorption inhibitors, or a combination thereof.

17. (canceled)

18. A method for treating a viral infection in a host animal, the method comprising administering to the host animal a composition comprising a therapeutically effective amount of one or more compounds capable of targeting vWF, inhibiting vWF-platelet interaction, or both.

19. (canceled)

20. The method of any claim 16 further comprising administering to the host animal one or more angiotensin receptor blockers, one or more protease inhibitors, or a combination thereof.

21. The method of claim 16 further comprising administering to the host animal one or more vitamins, one or more antioxidants, or a combination thereof.

22. The method of claim 16 wherein the virus is an enveloped virus.

23. The method of claim 16 wherein the virus is a RNA virus.

24. The method of claim 16 wherein the virus is positive-sense virus.

25. The method of claim 16 wherein the virus is a single stranded virus.

26. The method of claim 16 wherein the virus is a Coronaviridae.

27. The method of claim 16 wherein the virus is a Orthocoronavirinae.

28. The method of claim 16 wherein the virus uses, at least in part, host cell ACE2 receptors for entry.

29. The method of claim 16 wherein the virus uses, at least in part, an host cell serine protease for entry, for infection, or for both.

30. The method of claim 1 wherein the cholesterol-lowering agent is atorvastatin, simvastatin, or rosuvastatin, or a salt of the foregoing.

31. The method of claim 1 wherein the cholesterol absorption inhibitor is ezetimibe.

32. The method of claim 1 wherein the combination of cholesterol-lowering agents and cholesterol absorption inhibitor is a mixture of atorvastatin and ezetimibe.

33. The method of claim 2 further comprising administering to the host animal one or more angiotensin receptor blockers, one or more protease inhibitors, or a combination thereof.

34. The method of claim 2 further comprising administering to the host animal one or more vitamins, one or more antioxidants, or a combination thereof.

35. The method of claim 2 wherein the compound is caplacizumab.

36. The method of claim 2 wherein the composition further comprises one or more angiotensin receptor blockers, one or more protease inhibitors, or a combination thereof.

37. The method of claim 2 wherein the composition further comprises one or more vitamins, one or more antioxidants, or a combination of the foregoing.
